(19) United States
(12) Patent Application Publication
(10) Pub. No.: US 2010/0191060 A1
(43) Pub. Date: Jul. 29, 2010

(54) LIGHT GUIDE, LIGHT SOURCE APPARATUS AND ENDOSCOPE SYSTEM

(75) Inventor: Shinichi SHIMOTSU, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/693,073

(22) Filed: Jan. 25, 2010

(30) Foreign Application Priority Data
Jan. 27, 2009 (JP) 2009-014942

(51) Int. Cl. A61B 1/07; G02B 6/04; A61B 1/06

(52) U.S. Cl. 600/178; 362/554; 362/574

(57) ABSTRACT

Light from a light source enters a first small diameter fiber at an incident angle of 0°. Exit light from the first small diameter fiber has a substantially convex light intensity distribution in a diameter direction. Light from a light source enters a second small diameter fiber at an incident angle of 12°. Exit light from the second small diameter fiber has a substantially concave light intensity distribution in a diameter direction. The exit light from the first and second small diameter fibers enters a large diameter fiber via a fiber connector. Light inside the large diameter fiber has a substantially uniform light intensity distribution in a diameter direction with a light intensity not less than a predetermined value. The light is radiated as illumination light from a light exit section of the large diameter fiber.

### FIELD OF THE INVENTION

The present invention relates to a light guide for use in exposure of a semiconductor wafer and illumination of an endoscope. The present invention also relates to a light source apparatus and an endoscope system using this light guide.

### BACKGROUND OF THE INVENTION

Various optical fibers such as a bundle fiber, in which a plurality of optical fibers are bundled together, and a large diameter fiber having a diameter larger than a standard optical fiber are used for data signal communication. In addition, for example, such optical fiber is used as a light guide, in an exposure device for a semiconductor wafer, for guiding exposure light to a light exit section so as to expose the semiconductor wafer to the exposure light (see U.S. Pat. No. 7,059,778, corresponding to Japanese Patent Laid-Open Publication No. 2003-322730). In a light source apparatus of an endoscope, an optical fiber is used as a light guide which guides illumination light to a distal end of the endoscope so as to illuminate a body cavity of a patient (see Japanese Patent Laid-Open Publication No. 2000-199864).

In a case where the optical fiber is used as the light guide for guiding the exposure light as described in U.S. Pat. No. 7,059,778, a desired resist pattern cannot be produced if the radiation of light on the wafer is not uniform. In a case where the optical fiber is used as the light guide for illuminating the endoscope as described in Japanese Patent Laid-Open Publication No. 2000-199864, it becomes difficult to find a lesion if the light guided by the light guide has nonuniform light intensity distribution, and such light reflects off a region of interest having high reflectivity or uneven surfaces, because an image taken with the endoscope also becomes uneven in brightness.

Conventionally, to radiate light with a uniform light intensity distribution from a light guide, the number of optical fibers for forming a bundle fiber is increased. Alternatively or in addition, in U.S. Pat. No. 7,059,778, a position of exit light and its light intensity distribution are detected at a light exit surface of the optical fiber, and the light intensity distribution of the light incident on the optical fiber is controlled in accordance with the detection results. In Japanese Patent Laid-Open Publication No. 2000-199864, a light intensity distribution of exit light from an optical fiber is uniformized across its diameter direction by shifting a direction of a light incident end of the optical fiber to a direction vertical to an optical axis.

However, in U.S. Pat. No. 7,059,778, a device for detecting the position or the light intensity distribution of the exit light, or a device for controlling the light intensity distribution is required. In Japanese Patent Laid-Open Publication No. 2000-199864, a mechanism to shift the light incident end of the optical fiber is required. In either case, the light guide is upsized and additional cost is required for uniformization of the light intensity distribution.

Generally, in a case where light is incident on a multimode optical fiber through which light of various modes is propagated, or where multimode optical fibers are optically connected, light (laser) is input or the multimode optical fibers are optically connected at an angle not more than a numerical aperture (NA) of the optical fiber, namely, an acceptance angle of the optical fiber, in view of stabilizing the incident light or the connection of the multimode optical fibers. Accordingly, the exit light from the center portion of the multimode optical fiber has higher light intensity than the exit light from a peripheral portion thereof. Thus, the light intensity distribution at the light exit surface of the multimode optical fiber is not uniform.

### SUMMARY OF THE INVENTION

An object of the present invention is to provide a light guide, a light source apparatus and an endoscope system for uniformizing a light intensity distribution of exit light without cost and without upsizing the apparatus.

In order to achieve the above objects and other objects, a light guide of the present invention includes a first multimode optical fiber, a second multimode optical fiber and a bundling section. Light is incident on the first multimode optical fiber such that exit light from the first multimode optical fiber has a convex light intensity distribution having high light intensity in its center portion in a diameter direction of the first multimode optical fiber. Light is incident on the second multimode optical fiber such that exit light from the second multimode optical fiber has a concave light intensity distribution having low light intensity in its center portion in a diameter direction of the second multimode optical fiber. The bundling section bundles at least light exit surface sides of the first and second multimode optical fibers to form a bundle surface of a bundle fiber.

It is preferable that an incident angle of the light on the second multimode optical fiber is larger than an incident angle of the light on the first multimode optical fiber. It is preferable that each of the first and the second multimode optical fibers has an acceptance angle θ, and the incident angle of the light on the first multimode optical fiber is not less than 0° and not more than θ/2, and the incident angle of the light on the second multimode optical fiber is not less than θ/2 and not more than θ.

It is preferable that an inclination angle of a light incident surface of the second multimode optical fiber is larger than an inclination angle of a light incident surface of the first multimode optical fiber. It is preferable that each of the first and the second multimode optical fibers has an acceptance angle θ, and the inclination angle of the first multimode optical fiber is not less than 0° and not more than θ/2, and the inclination angle of the second multimode optical fiber is not less than θ/2 and not more than θ.

It is preferable that the light guide of the present invention further includes a third multimode optical fiber optically connected to the bundle fiber. The third multimode optical fiber has a light incident surface facing the bundle surface. The light incident surface is larger than the bundle surface in diameter. The light intensity distribution of the exit light from the first and second multimode optical fibers is further uniformized in the third multimode optical fiber.

It is preferable that the light guide further includes a speckle reducer provided to the third multimode optical fiber.

The speckle reducer reduces speckle of the light to be output from the third multimode optical fiber.

It is preferable that a numerical aperture (NA) of each of the first, the second and the third multimode optical fibers is not less than 0.2. Light is incident on the first multimode optical fiber at an incident angle of not more than the acceptance angle, and with the NA significantly smaller than 0.2 so as to form the convex light intensity distribution of the exit light. On the other hand, light is incident on the second multimode optical fiber with the NA close to 0.2 so as to form the concave light intensity distribution of the exit light. Accordingly, in the present invention, the light intensity distribution is uniformized by fully utilizing the intrinsic NA of the optical fiber.

It is preferable that the total number of the first and the second multimode optical fibers is not more than 19. The present invention uniformizes the light intensity distribution without using a few hundreds of optical fibers as in the conventional apparatuses. Conventionally, uniformization of the light intensity distribution has been difficult unless a diameter of an optical fiber (an outer diameter of a protection layer of the optical fiber) is not less than 10 mm. The present invention, on the other hand, uniformizes the light intensity even if a diameter of each of the first and the second multimode optical fibers is not more than 1 mm.

A light source apparatus of the present invention includes at least a first light source and a second light source, a first multimode optical fiber, a second multimode optical fiber, a bundling section and a third multimode optical fiber. The first multimode optical fiber has a first light incident surface facing the first light source, and a first exit surface for outputting exit light of a convex light intensity distribution having high light intensity in its center portion in a diameter direction of the first multimode optical fiber. The first light incident surface is orthogonal to an optical path of the first light source. The second multimode optical fiber has a second light incident surface facing the second light source, and a second exit surface for outputting exit light of a concave light intensity distribution having low light intensity in its center portion in a diameter direction of the second multimode optical fiber. The second light incident surface is inclined relative to an optical path of the second light source. The bundling section bundles at least the first and the second exit surface sides of the first and the second multimode optical fibers to form a bundle surface of a bundle fiber. The third multimode optical fiber is optically connected to the bundle fiber. The third multimode optical fiber has a third light incident surface and a third exit surface. The third light incident surface is larger than the bundle surface in diameter. Illumination light is radiated from the third exit surface.

An endoscope system of the present invention includes a light source apparatus, an endoscope and an image processing apparatus. The endoscope has an image sensor. The image sensor takes an image of a body cavity illuminated with the illumination light from the third exit surface of the third multimode optical fiber. An image processing apparatus is connected to the endoscope. The processing apparatus processes a signal from the image sensor and forms an image.

According to the present invention, the light intensity distribution of the exit light is uniformized without additional cost and without upsizing the apparatus.

### BRIEF DESCRIPTION OF THE DRAWINGS

The above and other objects and advantages of the present invention will be more apparent from the following detailed description of the preferred embodiments when read in connection with the accompanied drawings, wherein like reference numerals designate like or corresponding parts throughout the several views, and wherein:

FIG. 1 is a schematic view of a light source apparatus of the first embodiment of the present invention;

FIG. 2A shows a curve of light intensity distribution of exit light from a small diameter fiber in a case where an incident angle is 0° (degree);

FIG. 2B shows an FFP (Far field pattern) of the exit light from the small diameter fiber of FIG. 2A;

FIG. 3A shows a curve of light intensity distribution of exit light from a small diameter fiber in a case where the incident angle is 12°;

FIG. 3B shows an FFP of the exit light from the small diameter fiber of FIG. 3A;

FIG. 4A shows a curve of light intensity distribution of exit light from a light exit section;

FIG. 4B shows an FFP of the exit light from the light exit section of FIG. 4A;

FIG. 5A shows a radiation pattern (FFP) of exit light from the small diameter fiber in a case where the incident angle is 0°;

FIG. 5B shows a radiation pattern (FFP) of exit light from the small diameter in a case where the incident angle is 12°;

FIG. 5C shows a radiation pattern (NFP) of exit light from the small diameter fiber in a case where the incident angle is 12°;

FIG. 5D shows a radiation pattern (FFP) on which the exit light shown in FIG. 5A and the exit light shown in FIG. 5B or Figure C are superimposed;

FIG. 6 is a schematic view of an endoscope system of the present invention;

FIG. 7 is a schematic view of a light source apparatus of the second embodiment of the present invention;

FIG. 8 is a schematic view of a light source apparatus of the third embodiment of the present invention;

FIG. 9 shows a curve of light intensity distribution (NFP) of exit light from the small diameter fiber in a case where the incident angle is 6°;

FIG. 10 shows a curve of light intensity distribution (NFP) of exit light from the small diameter fiber in a case where the incident angle is 8°;

FIG. 11 shows a curve of light intensity distribution (NFP) of exit light from the small diameter fiber in a case where the incident angle is 10°; and

FIG. 12 shows a curve of light intensity distribution (NFP) of exit light from the small diameter fiber in a case where the incident angle is 12°.

### DESCRIPTION OF THE PREFERRED EMBODIMENTS

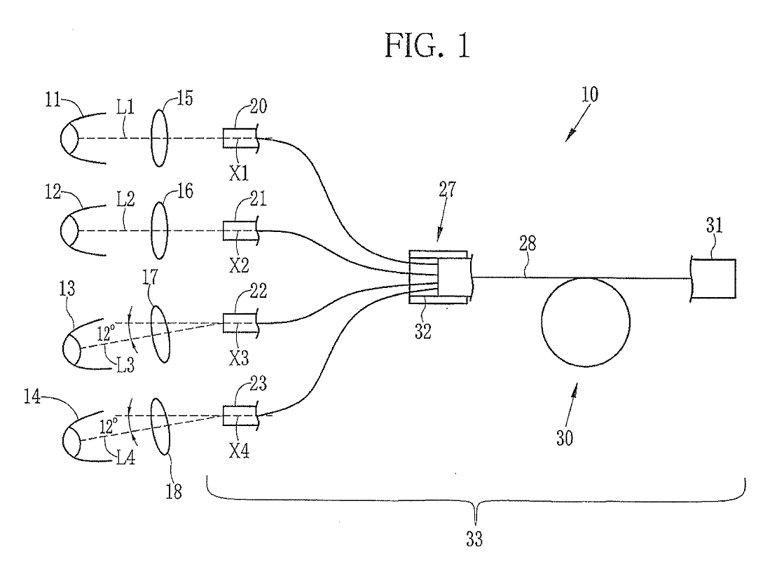

As shown in FIG. 1, a light source apparatus 10 of the first embodiment of the present invention has light sources 11 to 14, condenser lenses 15 to 18, small diameter optical fibers (hereinafter referred to as small diameter fibers) 20 to 23, a fiber connector 27 or bundling section or optical coupler, a large diameter optical fiber (hereinafter referred to as large diameter fiber) 28, a speckle reducer 30 and a light exit section 31 having an exit surface. The small diameter fibers 20 to 23 are bundled into a bundle fiber 32 using a ferrule or the like. A light guide 33 is composed of the bundle fiber 32 and the large diameter fiber 28. This light guide 33 guides light emitted from the light sources 11 to 14 to the light exit section 31. Only the ends of the small diameter fibers 20 to 23 on a light exit surface side may be bundled together. Alternatively, the entire small diameter fibers 20 to 23 may be bundled together.

In FIG. 1, light exit ends of the small diameter fibers 20 to 23 are depicted as lines. However, each light exit end actually has a rod-like shape as with the light incident end. The light exit ends of the small diameter fibers 20 to 23 are inserted into the sleeve-like fiber connector 27 and bundled. The large diameter fiber 28 is also inserted into the fiber connector 27. Thus, the bundle fiber 32 of the small diameter fibers 20 to 23 and the large diameter fiber 28 are optically connected. Any bundling device capable of bundling optical fibers may be used for bundling the small diameter fibers 20 to 23.

In a case where the bundle fiber 32 of the small diameter fibers 20 to 23 and the large diameter fiber 28 are aligned with high precision, a well-known ferrule structure is used as the bundling device. A through hole is formed at the center of each of first and second ferrules. The light exit ends of the small diameter fibers 20 to 23 are inserted together into the through hole of the first ferrule, and fixed inside the through hole with a transparent adhesive. A light incident end of the large diameter fiber 28 is inserted into the through hole of the second ferrule and fixed inside the through hole with the transparent adhesive. The first and second ferrules are inserted into a sleeve-like adaptor from opposite sides. Thus, the bundle fiber 32 of the small diameter fibers 20 to 23 and the large diameter fiber 28 are connected.

The light source 11 and the condenser lens 15 have a common optical axis L1. The light source 12 and the condenser lens 16 have a common optical axis L2. The optical axis L1 coincides with an optical axis X1 of the small diameter fiber 20. The optical axis L2 coincides with an optical axis X2 of the small diameter fiber 21. Accordingly, light emitted from the light source 11 enters the small diameter fiber 20 via the condenser lens 15 at an incident angle of 0° (degree). Light emitted from the light source 12 enters the small diameter fiber 21 via the condenser lens 16 at an incident angle of 0°. It should be noted that the incident angles to the small diameter fibers 20 and 21 (both with an acceptance angle θ) are not limited to 0°. The incident angles may be not less than 0° and not more than θ/2.

The light source 13 and the condenser lens 17 have a common optical axis L3. The light source 14 and the condenser lens 18 have a common optical axis L4. The optical axis L3 is tilted at 12° relative to an optical axis X3 of the small diameter fiber 22. The optical axis L4 is tilted at 12° relative to an optical axis X4 of the small diameter fiber 23. Accordingly, light emitted from the light source 13 enters the small diameter fiber 22 via the condenser lens 17 at the incident angle of 12°. Light emitted from the light source 14 enters the small diameter fiber 23 via the condenser lens 18 at the incident angle of 12°. It should be noted that the incident angles to the small diameter fibers 22 and 23 (both with the acceptance angle θ) are not limited to 12°. The incident angles may be not less than θ/2 and not more than θ. In a case where a numerical aperture (hereinafter abbreviated as NA) of each of the small diameter fibers 20 to 23 is 0.22, θis 12.7°.

Each of the small diameter fibers 20 to 23 and the large diameter fiber 28 is composed of a multimode optical fiber that propagates various modes of light. A diameter of the large diameter fiber 28 is larger than the diameter of the entire small diameter fibers 20 to 23 or the bundle fiber 32. Each of the small diameter fibers 20 to 23 and the large diameter fiber 28 is composed of a core, a clad surrounding the core and a protection layer covering the clad. An outer diameter of the large diameter fiber 28 including the protection layer is in a range from 2 mm to 40 mm. An outer diameter of the bundled small diameter fibers 20 to 23 or the bundle fiber 32 is in a range from 0.5 mm to 1.5 mm, and preferably 1 mm. The NA of the each of the small diameter fibers 20 to 23 is substantially the same as the NA of the large diameter fiber 28. Specifically, the NA is 0.2 or larger.

A core diameter of each of the small diameter fibers 20 to 23 is not less than 55 μm and not more than 65 μm, and more preferably 60 μm. A clad diameter of each of the small diameter fibers 20 to 23 is not less than 75 μm and not more than 85 μm, and more preferably 80 μm. A core diameter of the large diameter fiber 28 is not less than 225 μm and not more than 235 μm, and more preferably 230 μm. A clad diameter of the large diameter fiber 28 is not less than 245 μm and not more than 255 μm, and more preferably 250 μm.

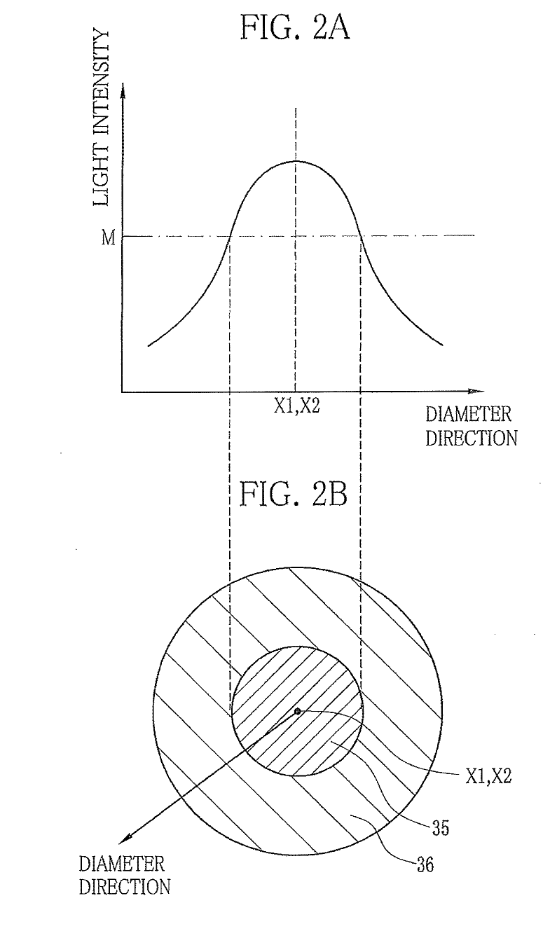

Each of the small diameter fibers 20 and 21 receives light at an incident angle of 0°. In FIG. 2A, each of the light intensity distributions in the small diameter fibers 20 and 21 is Gaussian, namely, a substantially convex or bell-shaped distribution having its peak on the optical axis X1 or X2. The light intensity decreases as a distance from the optical axis X1 or X2 increases. As shown in FIG. 2B, each of the far field patterns (hereinafter abbreviated as FFP) of the exit light from the small diameter fibers 20 and 21 has an area 35 and an area 36. The area 35 having the light intensity not less than a predetermined value M is located within a predetermined distance from the optical axis X1 or X2 in the diameter direction of the small diameter fiber 20 or 21. The area 36 having the light intensity less than the predetermined value M is located outside the area 35. The light intensity distributions and the FFPs of the exit light at the incident angles in a range from 0° to 6° are substantially the same as those at the incident angle of 0°. In addition, two or more beams of light which differ in the light intensity distributions of the exit light may be incident on the small diameter fiber(s).

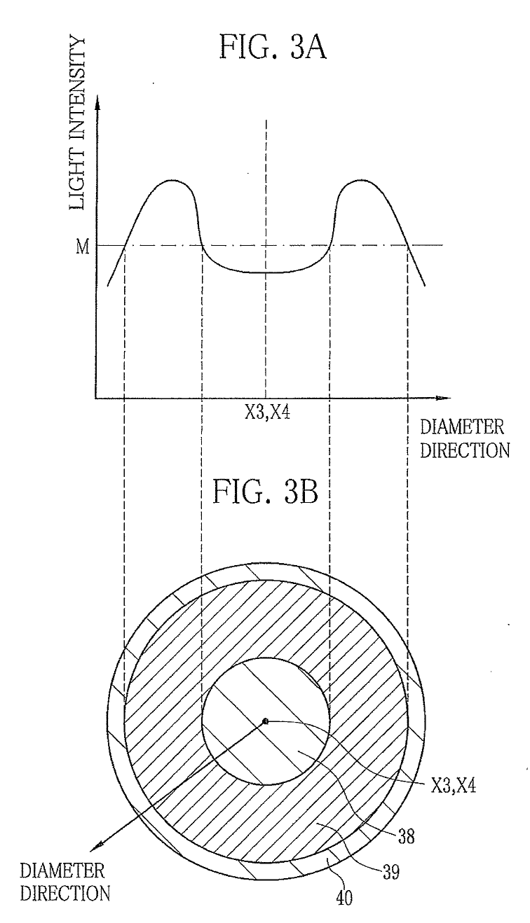

On the other hand, the small diameter fibers 22 and 23 receive light at the incident angle of 12°. As a result, as shown in FIG. 3A, each of the light intensity distributions of the small diameter fibers 22 and 23 shows a substantially concave curve (an annular radiation pattern) in which the light intensity of a center portion containing the optical axis X3 or X4 is smaller than the light intensity of its peripheral portion in the diameter direction. As shown in FIG. 3B, each of the FFPs of the exit light from the small diameter fibers 22 and 23 has an area 38, an area 39 and an area 40. The area 38 is located within a predetermined distance from the optical axis X3 or X4 in the diameter direction of the small diameter fiber 22 or 23 and has the light intensity less than the predetermined value M. The area 39 surrounds the area 38 and has the light intensity not less than the value M. The area 40 surrounds the area 39 and has the light intensity less than the value M.

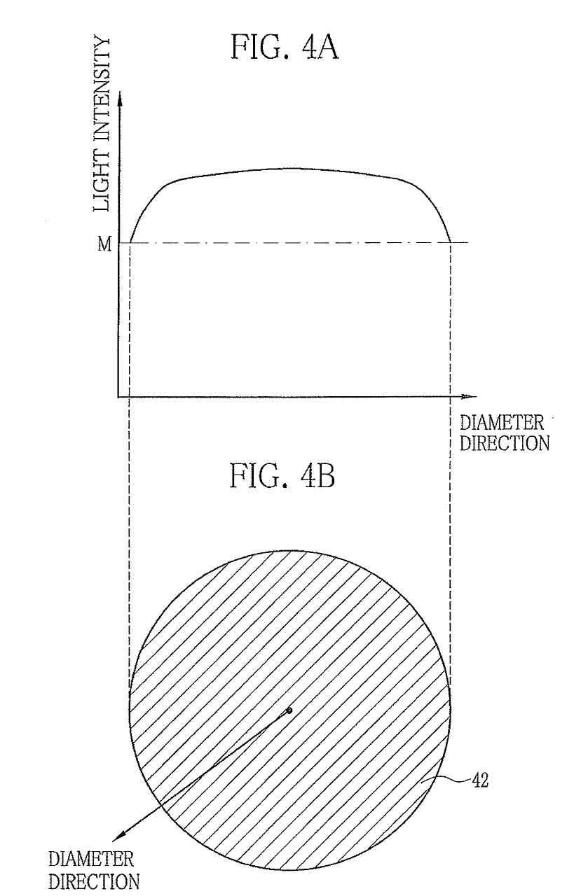

As shown in FIG. 1, the fiber connector 27 connects a light exit surface or bundle surface of the bundled small diameter fibers 20 to 23 or bundle fiber 32, and a light incident surface of the large diameter fiber 28 via a protection medium (not shown). The exit light from the small diameter fibers 20 to 23 enters the large diameter fiber 28. In the large diameter fiber 28, the exit light from the small diameter fibers 20 and 21 each having the substantially convex light intensity distribution, and the exit light from the small diameter fibers 22 and 23 each having substantially concave light intensity distribution are superimposed or combined. Thereby, as shown in FIG. 4A, the exit light from the large diameter fiber 28 has a substantially uniform flat-top light distribution with the light intensity not less than a predetermined value M across its diameter direction. As shown in FIG. 4B, an entire area 42 of the FFP of the exit light from the large diameter fiber 28 has the light intensity not less than the value M.

In the speckle reducer 30, the large diameter fiber 28 with several turns is vibrated to reduce speckle noise, further uniformizing the light intensity distribution. Thereby, the exit light with more uniform light intensity distribution is radiated from the light exit section 31. As a result, occurrence of the speckle noise is reduced. The light exit section 31 radiates the light to an object to be illuminated such as a screen.

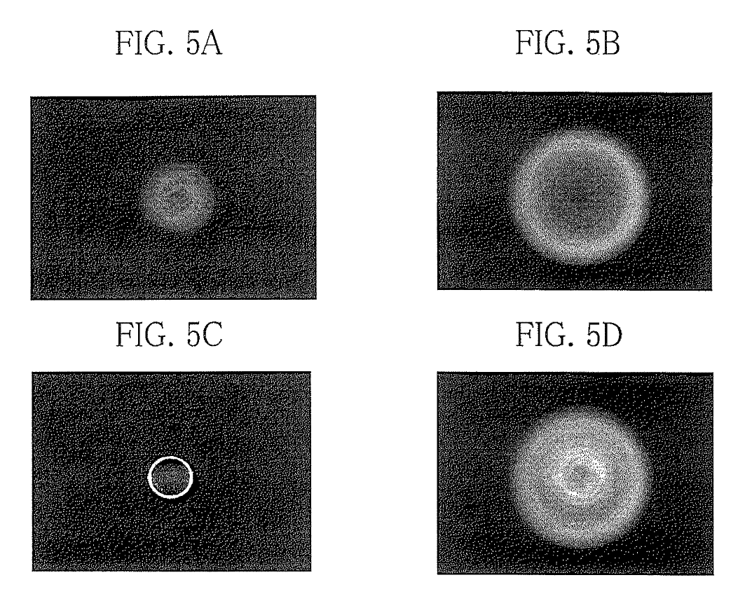

FIG. 5A shows the FFP of the exit light radiated on a screen from each of the light exit surfaces of the small diameter fibers 20 and 21 on which light is incident at the incident angle of 0°. White portions indicate where the light intensity is high. FIG. 5B shows the FFP of the exit light radiated on the screen from each of the light exit surfaces of the small diameter fibers 22 and 23 on which light is incident at the incident angle of 12°. FIG. 5C is a near field pattern (hereinafter abbreviated as NFP) of the exit light from each of the small diameter fibers 22 and 23 at the light exit surfaces thereof. FIG. 5D shows the radiation pattern of the exit light radiated on the screen from the exit surface of the light exit section 31 of the large diameter fiber 28 in a case where the light having the radiation pattern of FIG. 5A and the light having the radiation patterns of FIG. 5B and FIG. 5C is output to the large diameter fiber 28. FIG. 5D shows that the light intensity distribution of the exit light from the light exit section 31 is substantially uniform.

As described above, in the present invention, the light is incident on the small diameter fibers 20 and 21 such that the substantially convex light intensity distributions are formed, and the light is incident on the small diameter fibers 22 and 23 such that the substantially concave light intensity distributions are formed. The light having the substantially convex light intensity distributions and the light having the substantially concave light intensity distributions are superimposed. Thus, the light intensity distribution of the exit light from the light exit section 31 is uniformized.

The present invention makes the light intensity distribution uniform without specific devices described in U.S. Pat. No. 7,059,778 and Japanese Patent Laid-Open Publication No. 2000-199864. Accordingly, the apparatus of the present invention is prevented from upsizing, and does not require additional cost. Conventionally, after the replacement of the bundle fiber or the entire light guide, readjustments of control systems of the apparatuses for uniformizing the light intensity distribution are necessary. The present invention, on the other hand, only needs to set incident angles of the small diameter fibers 20 to 23. As a result, the time required for replacing the bundle fiber or the entire light guide is shortened compared with the conventional apparatuses. The present invention is particularly effective in cases where the light guide is frequently replaced, such as the light guide for illumination provided in an endoscope.

Conventionally, at least a few hundreds of optical fibers are necessary to make the light intensity distribution of a bundle fiber uniform due to the increase in the number of the optical fibers bundled in the bundle fiber. The present invention, on the other hand, requires at least two and at most 19 optical fibers to make the light intensity distribution uniform. Since the NA of each of the small diameter fibers 20 to 23 and the large diameter fiber 28 is not less than 0.2, the light intensity in a peripheral portion in the diameter direction of the large diameter fiber 28 is further increased. In a case where the light intensity in the peripheral portion is not large enough, the light intensity distribution is made uniform by superimposing light having a substantially concave light intensity distribution with increased light intensity in the peripheral portion.

Although the small diameter fiber and the large diameter fiber differ in diameter, the radiation pattern, for example, an annular radiation pattern, of the exit light from the small diameter fiber maintains its size and shape in the large diameter fiber. Conventionally, it is difficult to make the light intensity distribution uniform unless the diameter (outer diameter of the protection layer) of the optical fiber is at least 10 mm. The present invention, however, makes the light intensity distribution uniform even if the diameter of the small diameter fiber is not more than 1 mm.

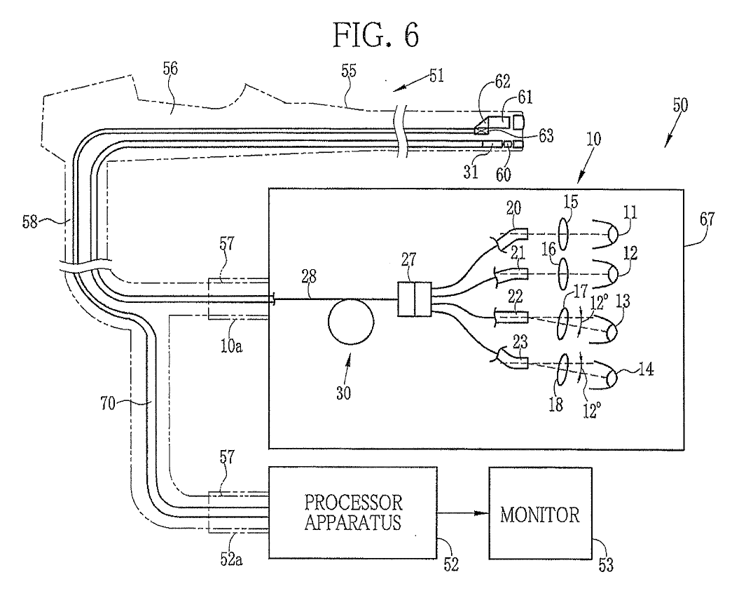

As shown in FIG. 6, an endoscope system 50 uses the light source apparatus 10 of the present invention as an apparatus for generating illumination light to illuminate a body cavity of a patient. An image of the body cavity of the patient illuminated with the illumination light is taken with an endoscope 51. A processor apparatus 52 or image processing apparatus performs various processing to the taken image. Thereafter, the image is displayed on a monitor 53.

The endoscope 51 is provided with a flexible insert section 55 to be inserted in a body cavity of a patient, a handling section 56 provided at a base portion of the insert section 55 and used for operating the endoscope 51 with a hand, and a universal cord 58 for connecting universal connectors 57 and the handling section 56. The universal connectors 57 are connected to a socket 10a of the light source apparatus 10 and a socket 52a of the processor apparatus 52, respectively. In a distal end of the insert section 55, an illumination optical system 60, an objective optical system 61, a prism 62 and an image sensor 63 are provided.

In a casing 67 are provided the light sources 11 to 14, the condenser lenses 15 to 18, the small diameter fibers 20 to 23, the fiber connector 27, and the speckle reducer 30 of the light source apparatus 10. An end portion of the large diameter fiber 28 is located inside the casing 67, and extends through the universal cord 58 and the insert section 55.

The light from the light source 11 is incident on the small diameter fiber 20 at an incident angle of 0° via the condenser lens 15. The light from the light source 12 is incident on the small diameter fiber 21 at an incident angle of 0° via the condenser lens 16. The exit light from each of the small diameter fibers and 21 has the substantially convex light intensity distribution curve shown in FIG. 2A and the FFP shown in FIG. 2B. The light from the light source 13 is incident on the small diameter fiber 22 at an incident angle of 12° via the condenser lens 17. The light from the light source 14 is incident on the small diameter fiber 23 at an incident angle of 12° via the condenser lens 18. The exit light from each of the small diameter fibers 22 and 23 has the substantially concave light intensity distribution curve shown in FIG. 3A and the FFP shown in FIG. 3B.

The exit light from the small diameter fibers 20 to 23 is output to the large diameter fiber 28 via the fiber connector 27. As shown in FIG. 4A, the light intensity distribution of the light inside the large diameter fiber 28 is substantially uniform with the light intensity not less than the predetermined value M across its diameter direction. In addition, as shown in FIG. 4B, the entire area 42 of the FFP of the large diameter fiber 28 has the light intensity not less than the predetermined value M. The light intensity distribution of the light inside the large diameter fiber 28 is further uniformized by the speckle reducer 30 and transmitted to the illumination optical system 60.

The illumination optical system 60 irradiates the body cavity of the patient with the light transmitted from the large diameter fiber 28. Since the illumination light has the uniform light intensity, an image obtained with the endoscope 51 is sharp even if a region of interest in the body cavity has a high reflectivity or significantly uneven surfaces. As a result, it becomes easy to find a lesion in the acquired image.

The objective optical system 61 receives light reflected off the region of interest in the body cavity. The prism 62 refracts the received light. An image is formed on an imaging surface of the image sensor 63 by the refracted light. Thereby, image signals of the region of interest are obtained. The obtained image signals are transmitted to the processor apparatus 52 via the insert section 55 and a signal line 70 in the universal cord 58. The processor apparatus 52 performs various processing to the image signals transmitted through the signal line 70. The monitor 53 displays an image of the region of interest based on the processed image signals.

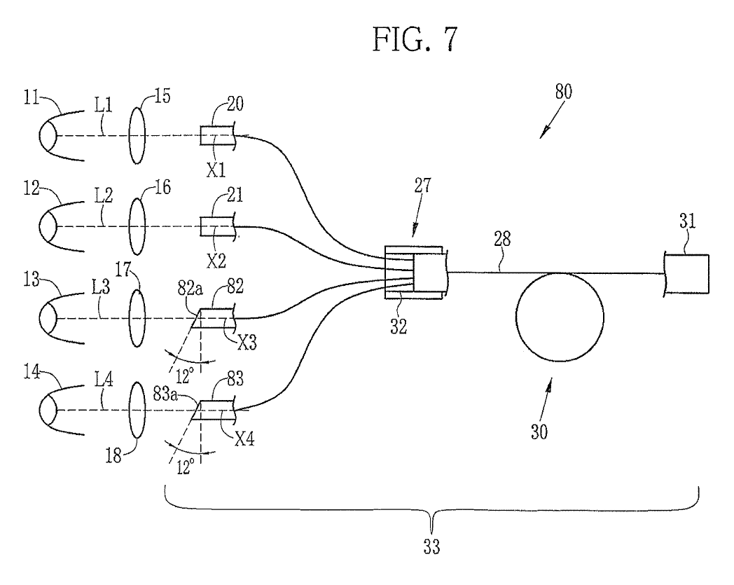

As shown in FIG. 7, a light source apparatus 80 of the second embodiment of the present invention has the same configuration as the light source apparatus 10 of the first embodiment shown in FIG. 1 except for small diameter fibers 82 and 83 each having an acceptance angle θ. The light source 13 and the condenser lens 17 have the common optical axis L3. The light source 14 and the condenser lens 18 have the common optical axis L4. The optical axis L3 coincides with an optical axis X3 of the small diameter fiber 82. The optical axis L4 coincides with an optical axis X4 of the small diameter fiber 83. Light incident surfaces 82a and 83a of the small diameter fibers 82 and 83 are ground or polished so as to be inclined 12° relative to planes orthogonal to the optical axes X3 and X4, respectively. In a case where each of the small diameter fibers 82 and 83 has the acceptance angle θ, each of the inclination angles of the light incident surfaces 82a and 83a of the small diameter fibers 82 and 83 may be not less than θ/2 and not more than θ relative to the plane orthogonal to the optical axes X3 or X4. In addition, each of the light incident surfaces of the small diameter fibers 20 and 21 may be ground or polished so as to be inclined at an inclination angle smaller than those of the light incident surfaces 82a and 83a of the small diameter fibers 82 and 83. The inclination angles of the light incident surfaces of the small diameter fibers 20 and 21 may be, for example, not less than 0° and not more than θ/2 relative to a plane orthogonal to the optical axis X1 or X2 in a case where each of the small diameter fibers 20 and 21 has the acceptance angle θ.

The small diameter fibers 82 and 83 are multimode optical fibers as with the small diameter fibers 20 and 21. Accordingly, when the light from the light sources 13 and 14 enters the light incident surfaces 82a and 83a inclined at the angle of 12° via the condenser lenses 17 and 18, respectively, the exit light from each of the small diameter fibers 82 and 83 has the substantially concave light intensity distribution shown in FIG. 3A and the FFP shown in FIG. 3B.

The exit light from the small diameter fibers 20, 21, 82 and 83 enters the large diameter fiber 28 through the fiber connector 27. In the large diameter fiber 28, the exit light from the small diameter fibers 20, 21, 82 and 83 is superimposed and uniformized. Thereby, as shown in FIG. 4A, the exit light from the large diameter fiber 28 has the substantially uniform light intensity distribution with the light intensity not less than the predetermined value M across its diameter direction. As shown in FIG. 4B, the entire area 42 of the FFP of the large diameter fiber 28 has the light intensity not less than the predetermined value M. The light intensity distribution of the light inside the large diameter fiber 28 is further uniformized by the speckle reducer 30.

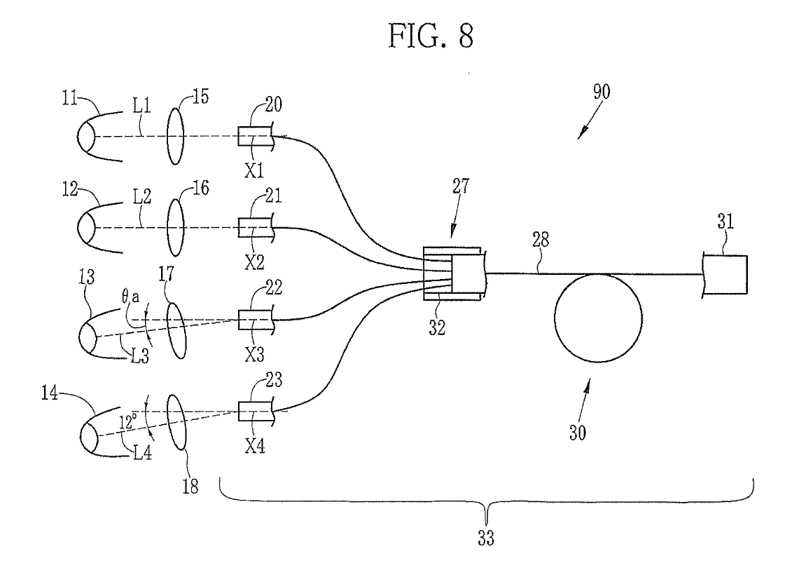

As shown in FIG. 8, a light source apparatus 90 of the third embodiment of the present invention has the same configuration as the light source apparatus 10 of the first embodiment shown in FIG. 1 except for an incident angle θa of the small diameter fiber 22. In this embodiment, the incident angle θ a is changeable within a range from 0° to 12°.

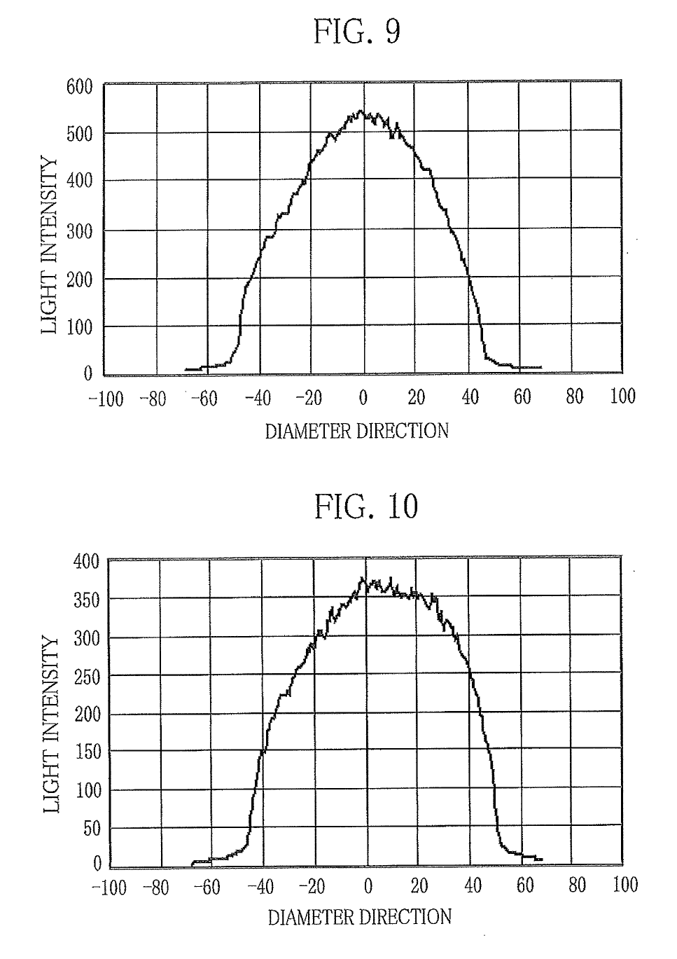

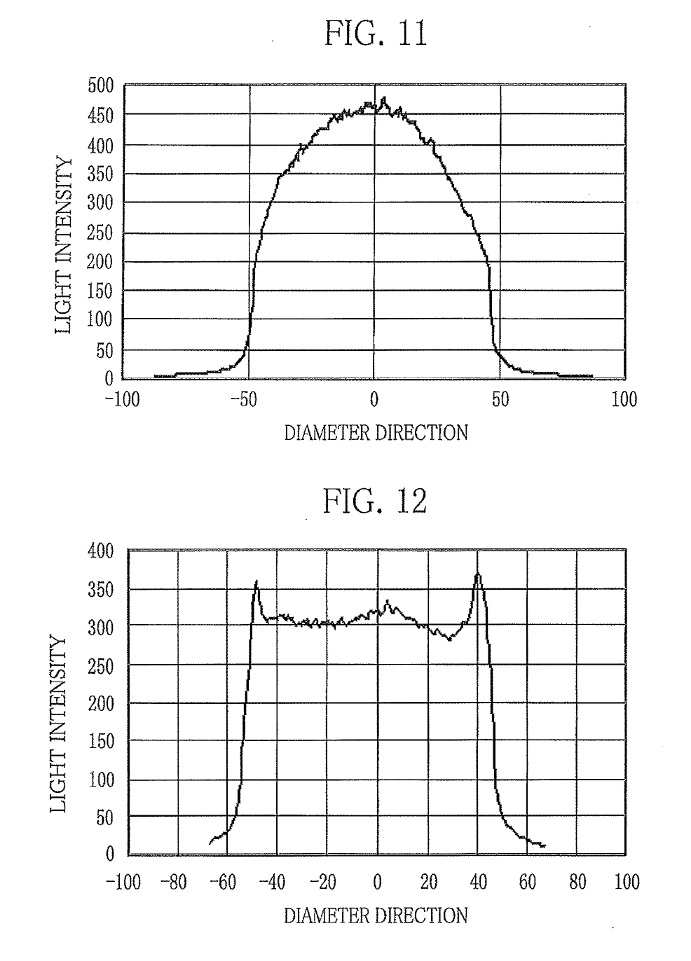

In FIGS. 9 to 12, the small diameter fiber 22 has the core diameter of 60 μm, the clad diameter of 80 μm, and the NA of 0.23. Each of FIGS. 9 to 12 shows a curve of the light intensity distribution (NFP) of the exit light from the small diameter fiber 22. In FIG. 9, the incident angle θa is 6°. In FIG. 10, the incident angle θa is 8°. In FIG. 11, the incident angle θa is 10°. In FIG. 12, the incident angle θa is 12°. In FIGS. 9 to 12, “0” in the “diameter direction” (horizontal axis) indicates the optical axis of the small diameter fiber 22. To form an annular radiation pattern, it is preferable to set the NA of the optical fiber close to its upper limit.

As shown in FIGS. 9 to 12, the light intensity in the peripheral portion of the small diameter fiber 22 in the diameter direction increases as θa increases from around 8°. It is known that the radiation pattern on the light exit surface of the small diameter fiber 22 changes in shape as θa changes, for example, from an annular-shape to an elliptical-shape and vice versa. Particularly, in a case where θa is 12°, the NA reaches the upper limit (0.22) of the optical fiber. Thereby, mode excitation in the peripheral portion of the radiation pattern becomes remarkable. Accordingly, the radiation pattern on the light exit surface of the small diameter fiber 22 becomes an annular-shape in a case where θa is 12°, significantly different from the radiation patterns with θa of less than 12°. In a case where 8a is in a range from 0° to 6°, the light intensity distribution of the exit light from the small diameter fiber 22 has substantially the same pattern (see FIG. 9) as in the case where θa is 6°.

Through the fiber connector 27, the exit light from the small diameter fiber 22 whose incident angle is θa enters the large diameter fiber 28 together with the exit light from the small diameter fibers 20 and 21 whose incident angles are 0°, and the exit light from the small diameter fiber 23 whose incident angle is 12°. Inside the large diameter fiber 28, the light output from the small diameter fibers 20 to 23 are superimposed with each other, making the light intensity uniform across the diameter direction of the large diameter fiber 28.

In a case where the incident angle θa of the small diameter fiber 22 is different from the incident angle 12° of the small diameter fiber 23, light having various radiation patterns different in size and shape enters the large diameter fiber 28, and is superimposed with each other in the large diameter fiber 28. Thereby, the exit light output from the exit surface of the light exit section 31 has the radiation pattern in which the multiple radiation patterns different in size and shape are combined and in which the light intensity distribution is uniform. In other words, the light with the desired radiation pattern can be radiated to the object to be illuminated by adjusting the incident angle θa of the small diameter fiber 22. The light incident on the small diameter fibers 20 and 21 is gathered along and close to the optical axes X1 and X2 with the use of the condenser lenses 15 and 16, respectively, so the light intensity becomes insufficient in the peripheral portion of the large diameter fiber 28 in the diameter direction. However, the light intensity in the peripheral portion is increased by the adjustment of the incident angle θa of the light incident on the small diameter fiber 22 while the uniformity in the light intensity distribution of the large diameter fiber 28 is not disturbed.

In the above embodiments, the small diameter fibers or bundle fiber and the large diameter fiber are connected, and the exit light is radiated from the large diameter fiber. Alternatively, the exit light may be directly radiated from the small diameter fibers without the use of the large diameter fiber. In this case, it is preferable to use a bundle fiber of two wraps formed as described in the following. First, a small diameter fiber is wrapped in a first protection tube, which is used as a center fiber of the bundle fiber. Multiple small diameter fibers are disposed around the center fiber, and they are wrapped in a second protection tube. Around the second protection tube, other multiple small diameter fibers are disposed, and they are wrapped in a third protection tube. Thus, the bundle fiber of two wraps is formed. It should be noted that such bundle fiber or composite fiber of two or more wraps may be used.

Various changes and modifications are possible in the present invention and may be understood to be within the present invention.

What is claimed is:

1. Alight guide for transmitting light for illumination, comprising:
a first multimode optical fiber on which said light is incident such that exit light from said first multimode optical fiber has a convex light intensity distribution having high light intensity in its center portion in a diameter direction of said first multimode optical fiber;
a second multimode optical fiber on which said light is incident such that exit light from said second multimode optical fiber has a concave light intensity distribution having low light intensity in its center portion in a diameter direction of said second multimode optical fiber; and
a bundling section for bundling at least light exit surface sides of said first and second multimode optical fibers to form a bundle surface of a bundle fiber.

2. The light guide of claim 1, wherein an incident angle of said light on said second multimode optical fiber is larger than an incident angle of said light on said first multimode optical fiber.

3. The light guide of claim 2, wherein each of said first and said second multimode optical fibers has an acceptance angle 8, and said incident angle of said light on said first multimode optical fiber is not less than 0° and not more than θ/2, and said incident angle of said light on said second multimode optical fiber is not less than θ/2 and not more than θ.

4. The light guide of claim 1, wherein an inclination angle of a light incident surface of said second multimode optical fiber is larger than an inclination angle of a light incident surface of said first multimode optical fiber.

5. The light guide of claim 4, wherein each of said first and said second multimode optical fibers has an acceptance angle θ, and said inclination angle of said first multimode optical fiber is not less than 0° and not more than θ/2, and said inclination angle of said second multimode optical fiber is not less than θ/2 and not more than θ.

6. The light guide of claim 1, further comprising a third multimode optical fiber optically connected to said bundle fiber, said third multimode optical fiber having a light incident surface facing said bundle surface, said light incident surface being larger than said bundle surface in diameter.

7. The light guide of claim 6, further comprising a speckle reducer provided to said third multimode optical fiber, said speckle reducer reducing speckle of said light to be output from said third multimode optical fiber.

8. The light guide of claim 7, wherein a numerical aperture of each of said first, said second and said third multimode optical fibers is not less than 0.2.

9. The light guide of claim 1, wherein a total number of said first and said second multimode optical fibers is at most 19.

10. The light guide of claim 1, wherein a diameter of each of said first and said second multimode optical fibers is not more than 1 mm.

11. A light source apparatus comprising:
at least a first light source and a second light source;
a first multimode optical fiber having a first light incident surface facing said first light source, and a first exit surface for outputting exit light of a convex light intensity distribution having high light intensity in its center portion in a diameter direction of said first multimode optical fiber, said first light incident surface being orthogonal to an optical path of said first light source;
a second multimode optical fiber having a second light incident surface facing said second light source, and a second exit surface for outputting exit light of a concave light intensity distribution having low light intensity in its center portion in a diameter direction of said second multimode optical fiber, said second light incident surface being inclined relative to an optical path of said second light source;
a bundling section for bundling at least first and second exit surface sides of said first and said second multimode optical fibers to form a bundle surface of a bundle fiber; and
a third multimode optical fiber optically connected to said bundle fiber, said third multimode optical fiber having a third light incident surface and a third exit surface, said third light incident surface being larger than said bundle surface in diameter, illumination light being radiated from said third exit surface.

12. An endoscope system comprising:
A. a light source apparatus including:
at least a first light source and a second light source;
a first multimode optical fiber having a first light incident surface facing said first light source, and a first exit surface for outputting exit light of a convex light intensity distribution having high light intensity in its center portion in a diameter direction of said first multimode optical fiber, said first light incident surface being orthogonal to an optical path of said first light source;
a second multimode optical fiber having a second light incident surface facing said second light source, and a second exit surface for outputting exit light of a concave light intensity distribution having low light intensity in its center portion in a diameter direction of said second multimode optical fiber, said second light incident surface being inclined relative to an optical path of said second light source;
a bundling section for bundling at least first and second exit surface sides of said first and said second multimode optical fibers to form a bundle surface of a bundle fiber;
a third multimode optical fiber optically connected to said bundle fiber, said third multimode optical fiber having a third light incident surface and a third exit surface, said third light incident surface being larger than said bundle surface in diameter;
B. an endoscope having an image sensor, said image sensor taking an image of a body cavity illuminated with illumination light from said third exit surface; and
C. an image processing apparatus connected to said endoscope, said processing apparatus processing a signal from said image sensor and forming an image.
